SEQUENCE LISTING (2 sequences)

SEQ ID NO: 1 - DNA, 2000 bp, Vibrio proteolyticus
  CDS (61)...(1890)
  Genomic DNA
tttaatttctgatttatcagtagttaaacaacgattgaaaataatctccaggattgagaaatgaataaaacacaacgtcacatcaactggctgctggctgttagcgcggcaactgcgctacctgtcaccgctgcagaaatgatcaacgtaaatgatggcagcctgctaaaccaggctcttaaagctcagtcacagagcgttgccccggtggaaaccggattcaaacaaatgaaacgagttgttttgccaaatggcaaagtgaaagttcgttatcaacaaactcaccacggtctaccggttttcaacacctcggtagtggcgactgaatcgaagtctggtagtagcgaagtgttaggtgtgatggctcagggtatcgcagacgacgtgtctacactgacgccatccgttgagatgaagcaggccatttcaattgctaaatcgcgtttccaacagcaagaaaaaatggttgcggaacctgcaacggaaaacgaaaaagccgagttgatggttcgtctggacgacaacaatcaagcgcaactagtgtatctggttgatttcttcgttgccgaggatcacccagcgcgtcctttctttttcattgatgcgcaaacgggtgaagtactgcaaacttgggatggtctgaaccatgcacaagctgacggtactggccctggcggtaacaccaaaacaggtcgttatgaatacggttctgactttcctccgtttgtcatcgataaagtcggcactaagtgttcaatgaacaacaccgcggtaagaacggttgacctgaacggctcaacttcaggtaacaccacttacagctatacctgtaacgactcaaccaactacaacgattacaaagccattaacggcgcgtactcgccactgaacgatgcccactacttcggtaaagtggttttcgatatgtacaaagactggatgaacaccacaccactgacgttccagctgactatgcgtgttcactatggtaacaactacgaaaacgcgttctggaatggttcatccatgaccttcggtgatggctacagcaccttctacccgctggtggatattaacgttagtgcccacgaagtgagccacggtttcaccgaacaaaactcgggtctggtgtacgagaatatgtctggtggtatgaacgaagcgttctctgatattgcaggtgaagcagcagagttctacatgaaaggcagcgttgactgggttgtcggtgcggatatcttcaaatcatccggcggtctgcgttactttgatcagccttcgcgtgacggccgttctatcgaccatgcgtctgactactacaatggcctgaatgttcactactcaagtggtgtattcaaccgtgcgttatacctgctggctaacaaagcgggttgggatgtacgcaaaggctttgaagtgtttaccctggctaaccaattgtactggacagcgaacagcacatttgatgaaggcggttgtggtgtagtgaaagctgcgagcgacatgggttacagcgttgcagacgtagaagatgcgtttaacacggtaggcgttaacgcgtcttgtggtgcaactcctcctccgtgtggcgatgtactggaaatcggtaaaccgctggcgaacctttcaggtaaccgcaatgacatgacttactacacgttcacaccaagcagctcatctagcgtagtgattaagatcactggcggtacaggtgatgcagacctttacgtgaaagcgggtagcaagccaaccacgacttcttacgattgccgtccatataagtatggtaacgaagagcagtgttcaatttcagcgcaagcgggtactacgtatcacgttatgctgcgtggttacagcaattacgctggtgtaactttgcgtgctgactaaactcagaatggaaccagtgaaggcgcaccttaaggtcgccttttttgtatcaggcgatctgtgtaaacgtgacctgatcgaagtgaggattggccgccagcgcttgcatg

SEQ ID NO: 2 - protein, 609 aa, Vibrio proteolyticus
MNKTQRHINWLLAVSAATALPVTAAEMINVNDGSLLNQALKAQSQSVAPVETGFKQMKRVVLPNGKVKVRYQQTHHGLPVFNTSVVATESKSGSSEVLGVMAQGIADDVSTLTPSVEMKQAISIAKSRFQQQEKMVAEPATENEKAELMVRLDDNNQAQLVYLVDFFVAEDHPARPFFFIDAQTGEVLQTWDGLNHAQADGTGPGGNTKTGRYEYGSDFPPFVIDKVGTKCSMNNTAVRTVDLNGSTSGNTTYSYTCNDSTNYNDYKAINGAYSPLNDAHYFGKVVFDMYKDWMNTTPLTFQLTMRVHYGNNYENAFWNGSSMTFGDGYSTFYPLVDINVSAHEVSHGFTEQNSGLVYENMSGGMNEAFSDIAGEAAEFYMKGSVDWVVGADIFKSSGGLRYFDQPSRDGRSIDHASDYYNGLNVHYSSGVFNRALYLLANKAGWDVRKGFEVFTLANQLYWTANSTFDEGGCGVVKAASDMGYSVADVEDAFNTVGVNASCGATPPPCGDVLEIGKPLANLSGNRNDMTYYTFTPSSSSSVVIKITGGTGDADLYVKAGSKPTTTSYDCRPYKYGNEEQCSISAQAGTTYHVMLRGYSNYAGVTLRAD

(19) United States
(12) Patent Application Publication
(10) Pub. No.: US 2005/0255095 A1
(43) Pub. Date: Nov. 17, 2005

(54) Destruction of prions using vibrolysin or variants thereof

(75) Inventor: Emil D. Kakkis, Novato, CA (US)

Correspondence Address: MARSHALL, GERSTEIN & BORUN LLP, 233 S. WACKER DRIVE, SUITE 6300, SEARS TOWER, CHICAGO, IL, 60606, US

(73) Assignee: BIOMARIN PHARMACEUTICAL INC., Novato, CA (US)

(21) Appl. No.: 11/058,555

(22) Filed: Feb. 15, 2005

Related U.S. Application Data
(63) Continuation of application No. 10/151,553, filed on May 16, 2002, now abandoned.
(60) Provisional application No. 60/291,665, filed on May 16, 2001.

(51) Int. Cl. A61K 38/48; G01N 33/53

(52) U.S. Cl. 424/94.63

(57) ABSTRACT

The present invention provides a method of reducing the activity of prions using vibriolysin or variants thereof. Vibriolysin-containing solutions are used to sanitize prion-contaminated facilities and instruments and decontaminate food products and biological tissues. The present invention provides a method of treating prion-related disease in animals and humans, comprising the administration of a formulation of vibriolysin or a variant thereof together with a pharmaceutically acceptable carrier. Such novel formulations are engineered to track the natural path of the prion from cells where the prions accumulate in the preclinical stage into neuronal cells and the brain at the advanced stage of the disease. The present invention provides methods and formulations that encompasses natural and recombinant vibriolysins and variants thereof with enhanced ability to access prion target cells, and with enzyme activity capable of being regulated by specific conditions, such as pH range or enzymatic cleavage.

### RELATED APPLICATIONS

This application is a non-provisional application derived from U.S. Provisional Application Ser. No. 60/291,665, filed May 16, 2001.

### TECHNICAL FIELD

This invention relates to a method of reducing the activity of infectious prions using vibriolysin or variants thereof. The present invention thereby provides a method of sanitization against prion-contaminated facilities and equipment and elimination of residual prions from prion-contaminated food products and biological tissues. The invention further provides a method of treatment of prion-related disease in animals and humans and novel formulations of compositions comprising vibriolysin.

### BACKGROUND OF THE INVENTION

In 1985 two reports described the purification of scrapie infectivity from an infected hamster brain, which comprised a fraction, highly enriched with a protein of an estimated Mr 28,000 to 30,000. The proteinacious infectivity entity was given the controversial designation “prion.” (McGeoch, et al., J. Gen. Virol. 67:813-830 (1986)) Prions have now been defined as small proteinacious infectious particles that resist inactivation by procedures which affect nucleic acids, and are composed mainly of a proteinacious infective entity, referred to as PrP^{Sc}. The PrP gene of mammals expresses a soluble protein, PrP^{C}, which can be converted to an insoluble form, PrP^{Sc}. Prion diseases result from the transformation of the normal form of prion protein (PrP^{C}) into the abnormal form (PrP^{Sc}) by a yet undefined post-translational event. PrP^{C} is bound to the external surface of cells by a glycoinositol phospholipid anchor, whereas PrP^{Sc} accumulates within cytoplasmic vesicles of cultured cells. Although there are no detectable differences between the amino acid sequences of the normal and disease forms of the prion protein, PrP^{Sc} has a conformation with a higher beta sheet and lower alpha-helix content (Gabizon and Prusiner, Biochem. J. 266:1-14 (1990); Prusiner, Science 252:1522 (1991); Prusiner, Acquired Immune Deficiency Syndromes 6(6):663-665 (1993); Pan, et al., Proc. Natl. Acad. Sci. USA 90:10962-10966 (1993); Safax, et al., J. Biol. Chem. 268:20276-20284 (1993)).

The presence of the abnormal PrP^{Sc} form in the brains of infected humans and animals is the only disease-specific diagnostic marker of prion disease. The PrP^{Sc} form mediates both the transmission and pathogenesis of prion diseases, including spongiform encephalopathies and plays a key role in neuronal degeneration (Prusiner, In: The Molecular and Genetic Basis of Neurological Disease, 2^{nd} Edition, pp. 103-143 (1997)). Prion diseases, such as scrapie, are characterized by abnormal processes of assembly and disassembly of normally soluble proteins into conformationally altered proteins in a defined, insoluble state. Other examples of insoluble proteins include the A beta peptide in amyloid plaques of Alzheimer's disease and cerebral amyloid angiopathy (CAA), alpha synuclein deposits in Lewy bodies of Parkinson's disease, Tau in neurofibrillary tangles in frontal temporal dementia and Pick's disease, superoxide dismutase in amyotrophic and lateral sclerosis, and huntington in Huntington's disease (Glenmer, et al., J. Neurol. Sci. 92:1-28 (1989); Haan, et al., Clin. Neurol. Neurosurg. 92(4):305-310 (1990)).

In addition to causing scrapie in sheep, prions are known to cause bovine spongiform encephalopathy (BSE) in cattle (also known as “mad cow disease”). In humans, prions also cause kuru disease in humans which cannibalizes the human brain, a genetically inherited form called Gerstmann-Straussler-Scheinker Syndrome (GSS), and forms of Creutzfeldt-Jakob disease (CJD) (Haseltine and Patarca, Nature 323(6084):115-116 (1986); Bazan, et al., Nature 325:581 (1987); Chatigny and Prusiner, Reviews Infectious Diseases 2(5):713-724 (1980); Prusiner, Science 216:136-144 (1982); McGeoch, et al., J. Gen. Virol. 67:813-830 (1986); Manuelidis, et al., Proc. Natl. Acad. Sci. USA 92:5124-5128 (1995); Prusiner, Science 278:245-251 (1997)). Recently, Supattapone, et al. (Mol. Cell. Biol. 21(7):2608-2616 (2001)) modified an abridged prion protein designated as PrP106 producing a 61-residue peptide, designated as PrP61. Transgenic mice expressing this PrP61 died spontaneously with ataxia and accumulated PrP61 within their neuronal dendrites and cell bodies.

Although prion infection directly by injection into the brain is most efficient in producing a pronounced disease state, oral ingestion of prions can also cause disease with lower frequency and prolonged duration of onset in animal models. Recently, it has become clear that the feeding of supplements comprising scrapie-infected sheep meat to cattle has led to infection of 177,490 animals in United Kingdom with a bovine form of prion disease (Donnelly, Nature 408:787-788 (2000)). The disease has spread to a lesser extent to France and other European countries. Furthermore, studies have demonstrated that prions from different species infect the same species most efficiently, but can cross a species barrier with decreased efficiency. It is now recognized that the human consumption of infected beef has led to a variant form of CJD (vCJD) in British citizens who consumed infected beef. It is possible that thousands of British and other European citizens are carrying prions in a preclinical state and will develop vCJD after some years of incubation. These findings, including the recent confirmation linking vCJD with BSE, therefore are the basis for the current concerns regarding the risks of acquiring human vCJD from eating infected beef (Roberts, et al., Curr. Biol. 6(10):1247-9 (1996); Collinge, Lancet 354(9175):317-323 (1999); Bruce, et al., Immunology Today 21:442-445 (2000); Donnelly, Nature 408:787-788 (2000)).

When prions enter the body via a peripheral route, they accumulate in the lymphoreticular tissues before moving through the nerves into the spinal cord or brain stem, and then to the brain. A hallmark of transmissible spongiform encephalopathies (TSEs) is the accumulation in nervous and lymphoid tissues of PrP^{Sc}. Prions ingested orally enter the body via follicular dendritic cells (FDCs) of the germinal centers within the reticuloendothelial system. Studies have shown that the FDCs of the germinal centers of the spleen, lymph nodes and Peyer's patches play an important role in the pathogenesis of transmissible spongiform encephalopathies (TSEs). These cells normally function to capture native antigen and present processed forms of antigens to B cells (Cardone and Pocchiari, Nature Medicine 7(4):410-411 (2001); Bruce, et al., Immunology Today 21:442-445 (2000)).

Studies have shown that the complement system is involved in the uptake of prions, replication in the lymphoreticular system and CNS invasion (Cardone and Pocchiari, Nature Medicine 7(4):410-411 (2001)). The complement system comprises serum proteins that can be activated by antibody-antigen complexes or the surface of microorganisms to undergo a cascade of proteolytic reactions resulting in the assembly of membrane attack complexes. Complement factors also enhance the ability of phagocytic cells to bind, ingest, and destroy the microorganisms subject to attack. In the normal humoral immune response, the antibody-antigen complex binds to Fc-gamma receptors and becomes covalently linked to complement adducts. These linked complexes then bind to the complement receptors to trigger antigen destruction. Depletion of either one of the early complement factors or the complement receptor significantly delays onset of disease symptoms in mice with scrapie, splenic accumulation of the pathological prion protein, and infectivity. Prions may bind to complement factors and the resulting prion-complement factor complex would then be captured by FDCs though complement receptors. Thus, the complement system may play an important role in opsonizing prion particles and enhancing complement receptor-mediated uptake into the FDCs. Prions in the FDCs may then incubate preclinically, increasing in concentration before being transported to the brain by FDCs or some type of white blood cell. (Cardone and Pocchiari, Nature Medicine 7(4):410411 (2001); Mabbott, et al., Nature Medicine 4(7):485-487 (2001); Klein, et al., Nature Medicine 7(4):488492 (2001)).

The properties of these protease resistant prions contribute to their stability and, hence, difficulty in their breakdown and elimination. Prions contain prion protein fragments of 208 amino acids in their murine form, though smaller fragments can generate prions. The prion protein contains two carbohydrate side chains and a glycophosphatidyl-inositol anchor, a lipid tail that inserts into the membrane. Prions are resistant to heat, detergent and to highly potent proteases such as proteinase K. The prion protein is very hydrophobic and forms P-pleated sheet structures, which make it difficult to digest. It is unclear whether autoclaving is sufficient to eliminate the prions. Given the risks of contacting and ingesting this very resistant particle, a method to reliably destroy or remove the prion, or counteract the adverse actions of prions, would be highly desirable (Supattapone, et al., Mol. Cell. Biol. 21(7):2608-2616 (2001); Chatigny and Prusiner, Reviews of Infectious Diseases 2(5):713-724 (1980)).

U.S. Pat. No. 6,214,366 B1 discloses a method of arresting, preventing, and/or reversing the impairment of physiologic systems by reducing the burden of insoluble protein deposits using branched polycationic agents, such as dendritic polycations, or pharmaceutical compositions containing such branched polycationic agents. The method of enhancing the clearance of PrP^{Sc} form from cells comprises administering a pharmaceutical composition comprising an unconjugated dendritic polycation with a pharmaceutically acceptable excipient. However, such a method may not be effective in the complete elimination of the infectious prion particles. U.S. Pat. No. 5,756,678 discloses a method for the treatment of connective tissue materials, such as collagen, for the inactivation of prions to obtain at least 5 logs of protection, comprising contacting the liquid solution of connective tissue material with sodium hydroxides that the sodium hydroxide in said solution ranges from 0.1 M-0.7 M for a period of time sufficient to inactivate the prions without adversely affecting the function of connective tissue at a temperature of 25° C. or less. This method is impractical for use in vivo and the requirement of sufficient exposure to high concentrations of sodium hydroxide may be damaging to some tissue transplants.

Schroder, et al., (Neurotoxicology 19(4-5):683-688 (1998)) studied mechanisms of prion^{Sc}-induced neuronal cell death and found that NMDA receptor antagonists blocked the effect of PrP^{Sc} to induce apoptosis in rat cortical neurons. In addition to inducing apoptosis, PrP106-126 caused a significant drop in the intracellular glutathione (GSH) level in neuronal cells. GSH together with the proto-oncogene product Bcl-2 protects neuronal cells against apoptotic cell death, possibly through lowering the load of reactive oxygen species (ROS) within cells. Thus, agents which lower the load of ROS in cells may counteract the apoptotic effects of prions. Supattapone, et al. (J. Virol. 75(7):3453-3461 (2001)) showed that branched polyamines in vitro disaggregated the prion rods, reduced the beta sheet content of PrP 27-30, and rendered PrP 27-30 susceptible to proteolysis in scrapie-infected neuroblastoma cells in culture. However, the susceptibility of PrP^{Sc} to proteolytic digestion induced by branched polyamines was strain-dependent, wherein PrP^{Sc} from bovine spongiform encephalopathy-infected brain but not PrP^{Sc} from natural sheep scrapie-infected brain were susceptible. Since the branched polyamines accumulated specifically in the lysosomes, this acidic compartment is believed to be the site where these agents mediate PrP^{Sc} clearance.

Vibriolysin is a proteolytic enzyme secreted by the Gram-negative marine microorganism, Vibrio proteolyticus. This endoprotease has specific affinity for the hydrophobic regions of proteins and is capable of cleaving proteins adjacent to hydrophobic amino acids. Since the interior portions of proteins are usually hydrophobic, vibriolysin is a potent protease useful in cleaving denatured and hydrophobic proteins and is active over a wide range of pH and temperature conditions (Durham, et al., J. Burn Care Rehabil. 14(5): 544-51 (1993)). The gene (nprV) which codes for the extracellular neutral protease, vibriolysin (NprV), was isolated from a V. proteolyticus DNA library constructed in Escherichia coli. The nucleotide sequence of the cloned nprV gene revealed an open reading frame encoding 609 amino acids including a putative signal peptide sequence followed by a long “pro” sequence consisting of 171 amino acids. The mature NprV purified from cultures of V. proteolyticus was compared to the sequences of the neutral proteases from Bacillus thermolyticus (thermolysin) and Bacillus stearthermophilus and extensive regions of conserved homology were identified, including active-site residues, zinc-binding residues and calcium-binding sites. (David, et al., Gene 112(1):107-112 (1992)). The DNA sequence of the vibriolysin gene (SEQ ID. NO. 1) is shown in FIG. 2. The DNA sequence shown comprises a portion of a 6.7 kb Hind III fragment of the Vibrio proteolyticus gene, which is described in U.S. Pat. Nos. 4,966,846 and 5,505,943. There is an open reading frame from approximately base 249-2078, within which the DNA region encoding vibriolysin is found. This DNA sequence encodes a protease isolated from the Vibrio strain Vibrio proteolyticus ATCC 53559. Such vibriolysin protease is known by several designations, including vibriolysin (DBSource pir: locus JT0903, EC 3.4.24.-) precursor—Vibrio proteolyticus; neutral protease precursor (Vibriolysin) (Aeromonolysin) (DBSource swissprot: locus NPRV_VIBPR, accession 000971, EC_number=“3.4.24.25”; and neutral protease [Vibrio proteolyticus] (DBSource locus VIBNEUP accession M64809.1) (David, et al., Gene 112:107-112 (1992)). Other vibriolysins have been previously described, and include: (1) virulence metalloprotease precursor (Vibriolysin) (Milton, et al., J. Bacteriol. 174(22):7235-7244 (1992); Norqvist, et al., Infect. Immun. 58(11):3731-3736 (1990)); (2) hemagglutinin/proteinase precursor (HA/Protease) (Vibriolysin) (Hase, et al., J. Bacteriol. 173(11):3311-3317 (1991); Heidelberg, et al., Nature 406(6795):477-483(2000); Hase, et al., Infect. Immun. 58(12):4011-4015 (1990)); (3) vibriolysin (EC 3.4.24.-) precursor [validated]—Vibrio cholerae (group O1 strain N16961) (Hase, et al., Infect. Immun. 58(12):4011-4015 (1990)); Hase, et al., J. Bacteriol. 173(11):3311-3317 (1991); Heidelberg, et al., Nature 406(6795):477-483 (2000)); (4) zinc metalloproteinase (EC 3.4.24.-) precursor—Legionella pneumophila (Black, et al., J. Bacteriol. 172(5):2608-2613 (1990)); (5) vibriolysin (EC 3.4.24.-) precursor—Vibrio vulnificus (Cheng, et al., Gene 183(1-2):255-257 (1996)); and (6) vibriolysin (EC 3.4.24.-) precursor—Vibrio anguillarum (Norqvist, et al., Infect. Immun. 58(11):3731-3736 (1990); Milton, et al., J. Bacteriol. 174(22):7235-7244 (1992)).

Vibriolysin has been shown to be particularly beneficial for debridement of burn wound eschar. In vitro experiments showed that vibriolysin was effective in hydrolyzing proteinacious components of eschar, including denatured fibrin, elastin and collagen. Vibriolysin exhibited desirable properties including selective hydrolysis of dead but not viable tissues, debridement in the absence of bleeding, compatibility with adjunct therapies and shelf-life stability in a hydrophilic composition at room temperature. Furthermore, vibriolysin was shown to stimulate granulation tissue or neodermis, and thereby, may have beneficial effects on dermal repair processes (Durham, et al., J. Burn Care Rehabil. 14(5):544-51 (1993); Nanney, et al., Wound Rep. Reg. 3:442-8 (1995)). U.S. Pat. Nos. 5,145,681 and 5,505,943 and International Application No. WO 98/55604 teach compositions for debriding wounds, comprising a pharmaceutically acceptable topical carrier admixed with an effective amount of a protease, which may be produced by: (1) a microorganism from the genus Vibrio, or (2) expression by recombinant host cells transformed or transfected with an expression vector for a protease produced by a microorganism from the genus Vibrio; or mutants and hybrids thereof. Unlike other proteolytic agents contemplated for use in debriding wounds and shown to be ineffective and toxic systemically and locally, vibriolysin was shown to have superior characteristics including debridement properties and the ability to promote wound healing.

It is now and herein postulated that because of the propensity of vibriolysins to degrade hydrophobic proteins, vibriolysin serves as an excellent candidate for a protease capable of destroying the prion protein by proteolytic cleavage of infectious prion particles. In other words, vibriolysin is characterized by properties consistent with the ability to degrade prions when the enzyme is formulated as a disinfectant solution for cleaning or sanitizing equipment and surfaces, or as a therapeutic for the treatment of prion-associated or prion-like diseases in animals and humans.

### SUMMARY OF THE INVENTION

One aspect of the invention relates to a composition capable of reducing the infectivity of prion, comprising vibriolysin or a variant thereof in an amount effective to reduce the infectivity of prion, and one or more cleaning adjunct materials. Another aspect of the invention relates to a DNA sequence encoding vibriolysin or a variant thereof, wherein said vibriolysin or a variant thereof has the ability to gain access to target sites that accumulate prions within the body.

Still another aspect of the invention relates to a method of reducing the activity of infectious prions, comprising the step of contacting a vibriolysin protease or variant thereof with said prions in an amount effective to cleave or degrade said prions or destroy their infective activity.

An important aspect of the invention relates to a method of sanitizing facilities or instruments contaminated with pirons comprising the step of contacting said prions with a solution comprising a vibriolysin protease or variant thereof in an amount effective to reduce or eradicate prion contamination of said instruments and facilities.

Yet another aspect of the invention relates to a method of treating or preventing a prion-caused, prion-related or prion-like diseases in a subject or biological tissue in need thereof comprising administering to said subject a formulation of a pharmaceutical composition comprising a vibriolysin protease or variant thereof with a pharmaceutically acceptable carrier in an amount effective to treat or prevent said prion-caused, prion-related or prion-like diseases.

In other words, the present invention provides a method of reducing the activity of prions using vibriolysin or a variant of the protease.

An important embodiment of the present invention provides a means of sanitizing actual or potentially prion-contaminated facilities and instruments. Yet another embodiment of the present invention is the decontamination of human and animal food products and nutritional supplements with formulations of vibriolysin to eliminate residual prions. Another embodiment is the decontamination of tissues used in human transplantation such as collagen tendons or bone grafts.

The present invention provides for a method of treating or preventing a prion-caused, prion-related or prion-like diseases in a subject or biological tissue in need thereof comprising: administering to said subject a formulation of a pharmaceutical composition comprising a vibriolysin protease or variant thereof with a pharmaceutically acceptable carrier in an amount effective to treat or prevent said prion-caused, prion-related or prion-like diseases.

More specifically, the present invention provides a method of treating prion-related diseases in animals and humans, including, but not limited to, scrapie, bovine spongiform encephalopathy (BSE), kuru disease, Gerstmann-Straussler-Scheinker Syndrome (GSS), and forms of Creutzfeld-Jacob (CJD) disease.

A preferred embodiment is a method of treating prion-related diseases at the preclinical stage, wherein prions incubating within follicular dendritic cells (FDCs) or other cells are cleaved by vibriolysin or variants thereof when taken up by the prion-infected cells. The method comprises administering a formulation of a pharmaceutical composition comprising vibriolysin or variant thereof, together with a pharmaceutically-acceptable carrier.

The present invention provides for novel formulations of vibriolysin and variants thereof, which enhance the capability of the enzyme to track the natural path of the prion from oral ingestion to cells where the prions accumulate in the preclinical stage to the movement of prions into neuronal cells and the brain at the advanced stage of the disease. Such novel formulations and various routes of administration are described. Another preferred embodiment is the treatment of prion-related diseases at the advanced stage, when prions have infected neuronal cells and the brain.

The present invention provides novel formulations of vibriolysin capable of transport across the blood brain barrier and uptake into neuronal cells and lysosomes where prions accumulate at the advanced stage.

The present invention also provides novel formulations of vibriolysin wherein its enzyme activity is regulated by changes in conditions such as pH or by exposure to proteolytic enzymes.

Another embodiment of the present invention is a DNA sequence encoding vibriolysin or a variant thereof engineered to enhance access of the enzyme to target sites of prions, such as uptake into cells (e.g. FDCs) or organelles (lysosomes) which accumulate prions, or transport across the blood brain barrier. Yet another embodiment of the present invention is a DNA sequence encoding vibriolysin or a variant thereof, engineered to transform from an inactive to active form upon exposure to specific conditions, such as a change in pH, temperature, chemical composition, or concentration, or to proteolytic enzymes.

### BRIEF DESCRIPTION OF THE FIGURES

FIG. 1 is a schematic representation of a formulation of vibriolysin and its administration and action. In this representation, active vibriolysin is portrayed as a cube, but vibriolysin may also be formulated in a pro- or inactive form.

1. The active Vibrio is aggregated or crystallized to form an accumulation of vibriolysin molecules, with the particle size dependent on the properties necessary for complement activation and also dependent on the necessary properties of stability and bioavailability. The vibriolysin in this aggregate is relatively inactive against proteins since such proteins are inaccessible to the enzyme in the crystal.

2. In order to assist in stabilizing and maintaining the aggregate for purposes of surviving other proteases and conditions in the digestive tract, the aggregate is cross-linked with a multi-crosslinking agent. For example, glutarylaldehyde 0.2% is a multi-crosslinking agent with a short period of exposure, for example. The crystal or aggregate does not readily redissolve at neutral pH.

3. The crosslinked aggregate or crystal is lipidated by attaching fatty acids or other similar hydrophobic chains to hydroxyls or other moieties exposed on the protein. Sufficient lipid is added to create a membrane-like surface which results in three features: (1) solubility of the enzyme in fats, thereby enhancing absorption into the lacteals and local lymph, whereby the fat containing the particle is absorbed similar to prion uptake; (2) creation, by lipidation of the particle, of the appearance of a foreign membrane to trigger the activation of complement and opsonization of the particle for uptake by white cells such as FDCs; and (3) maintenance of the enzyme in an inactive state within the lipid until the breakdown of the lipid layer by acid lipases within the lysosome.

4. The lipidated, crosslinked particle is administered orally in oil or within a fatty meal, wherein the vibriolysin particle enters the lymph system and is transported into lymph cells or the circulation. The lipid surface immediately activates complement.

5. The activated complement components binds and opsonizes the particle, which is then transported into FDCs or similar cells by complement receptor-mediated uptake.

6. The lipid coat is removed by the acid lipases or as a result of changes in pH, thereby exposing the crystal aggregate to the conditions of the lysosome.

7. The conditions within the lysosome trigger the disassembly of the aggregate and the slow dissolution of the vibriolysin.

8. The re-dissolved vibriolysin digests hydrophobic protein aggregates, and is formulated into a concentration and stability such that the enzyme activity diminishes over a specific duration.

Any part of the scheme may be modified or excluded to enhance the specific objective of reaching the site of prion storage, wherein the prion becomes activated. Activation of the prion remains restricted to such sites of prion storage.

FIG. 2 shows the DNA sequence of the vibriolysin gene (SEQ ID. NO. 1), and comprises a portion of a 6.7 kb Hind III fragment of the Vibrio proteolyticus gene, which is described in U.S. Pat. Nos. 4,966,846 and 5,505,943. There is an open reading frame from approximately base 249-2078, within which the DNA region encoding vibriolysin is found. This DNA sequence encodes a protease isolated from the Vibrio strain Vibrio proteolyticus ATCC 53559. This vibriolysin protease is known by several designations, including vibriolysin (DBSource pir: locus JT0903, EC 3.4.24.-) precursor—Vibrio proteolyticus; neutral protease precursor (Vibriolysin) (Aeromonolysin) (DBSource swissprot: locus NPRV_VIBPR, accession 000971, EC_number=“3.4.24.25”; and neutral protease (Vibrio proteolyticus) (DBSource locus VIBNEUP accession M64809.1) (David, et al., Gene 112:107-112 (1992)).

FIG. 3 depicts the digestion of prion protein derived from variant Creutzveld-Jacob Disease (vCJD) brain by vibriolysin. FIG. 3A depicts the X-ray film of a Western blot analysis of the digestion of prion containing vCJD brain with increasing concentrations of Vibriolysin as indicated at the temp of 20° C. FIG. 3B depicts the X-ray film of a Western blot analysis of the digestion of a prion extracts by Vibriolysin at the temp of 40° C. In both FIGS. 3A and 3B, the highest concentration of Vibriolysin reduced prion content about 10 fold after digestion for 1 hour. The conditions were not the optimal ones for Vibriolysin and therefore better digestion would be expected at higher pH and higher temp.

### DETAILED DESCRIPTION OF THE INVENTION

The present invention provides a method of reducing the infectious activity of prions using vibriolysin or a variant thereof. Preferably, the biologic activity of the infectious prions or the structure of the infectious prions is destroyed.

Vibriolysin is an endoprotease that is capable of cleaving proteins within hydrophobic regions. As a potent protease, vibriolysin can cleave denatured proteins and maintain activity over a wide range of pH and temperature conditions. Because of its propensity to degrade hydrophobic proteins, vibriolysin serves as an excellent candidate for a protease capable of destroying the prion protein by proteolytically cleaving the infectious prion particles. Given the wide range of conditions over which the protease is active, including extreme alkaline conditions (pH 10-14) in which most proteins are solubilized, it would be expected that vibriolysin could be formulated in a solution which would dissolve all proteins and proteolytically cleave the infectious prions.

An advantage of the present invention is that use of vibriolysin or variant thereof for cleansing and decontamination purposes is preferably over the use of chemicals, such as bleach, for reducing the infectivity or destroying infectious prion particles because the vibriolysin or variant thereof is itself not caustic or harmful to human and livestock.

The present invention also provides a protein variant of vibriolysin with activity and properties, which can degrade the prion protein. A variant of the vibriolysin protease encompasses endoproteases capable of: (I) cleaving proteins within hydrophobic regions and maintaining activity over a wide range of pH and temperature conditions; and (2) having an amino acid sequence comprising the active site residues. A variant can be naturally occurring or man-made. A list of naturally occurring vibriolysin variants is discussed above. A man-made variant vibriolysin can comprise substitutions of one or more amino acid of any naturally occurring vibriolysin variant. One skilled in the art can, using standard site-directed mutagenesis procedures, make a variant vibriolysin with an amino acid sequence that is not found in nature, and test its ability to cleave prion proteins (see Example). In this manner, amino acids that are essential (substitutions at amino acid positions resulting in the variants' complete ability to cleave prion proteins) and those that are non-essential (substitutions at amino acid positions resulting in the variants' increased, unaltered, or reduced, but not a complete reduction, ability to cleave prion proteins) can be determined. The present invention encompasses variant vibriolysins with one or more amino acid substitutions at non-essential amino acid positions. Similarly, using the method outlined above, the deletion of and/or insertion of amino acids into vibriolysin can determine other variants that retain activity to cleave prion proteins. Typically, the variant of the vibriolysin protease will have at least 90% homology, preferably at least 95%, and more preferably at least 98% homology to the natural vibriolysin protease.

The method of the present invention encompasses the use of natural vibriolysin produced and secreted by Vibrio and vibriolysin produced by recombinant methods and variants of both natural and recombinant vibriolysin thereof. Vibriolysin proteins which have been previously described include: (1) virulence metalloprotease precursor (Vibriolysin) (Milton, et al., J. Bacteriol. 174(22):7235-7244 (1992); Norqvist, et al., Infect. Immun. 58(11):3731-3736 (1990)); (2) hemagglutinin/proteinase precursor (HA/Protease) (Vibriolysin) (Hase, et al., J. Bacteriol. 173(11):3311-3317 (1991); Heidelberg, et al., Nature 406(6795):477-483(2000); Hase, et al., Infect. Immun. 58(12):4011-4015 (1990)); (3) vibriolysin (EC 3.4.24.-) precursor [validated]—Vibrio cholerae (group O1 strain N16961) (Hase, et al., Infect. Immun. 58(12):4011-4015 (1990)); Hase, et al., J. Bacteriol. 173(11):3311-3317 (1991); Heidelberg, et al., Nature 406(6795):477-483 (2000)); (4) zinc metalloproteinase (EC 3.4.24.-) precursor—Legionella pneumophila (Black, et al., J. Bacteriol. 172(5):2608-2613 (1990)); (5) vibriolysin (EC 3.4.24.-) precursor—Vibrio vulnificus (Cheng, et al., Gene 183(1-2):255-257 (1996)); (6) vibriolysin (EC 3.4.24.-) precursor—Vibrio anguillarum (Norqvist, et al., Infect. Immun. 58(11):3731-3736 (1990); Milton, et al., J. Bacteriol. 174(22):7235-7244 (1992)); (7) vibriolysin (EC 3.4.24.-) precursor—Vibrio proteolyticus (David, et al., Gene 112(1):107-112 (1992)); (8) neutral protease precursor (Vibriolysin) (Aeromonolysin) (David, et al., Gene 112(1):107-112(1992)); and (9) neutral protease [Vibrio proteolyticus] (David, et al., Gene 112:107-112 (1992)).

An important embodiment of the present invention provides for a method of sanitation of prion-contaminated facilities and/or instruments. For sanitizing solutions, the enzyme stability and activity of vibriolysin protease is highly desirable. For example, vibriolysin may be formulated in an alkaline solution of pH 10-14 which will assist in solubilizing proteins and lipids for cleaning and allow the destruction of prions effectively. Disinfectant solution comprising vibriolysin or variants thereof can be used to sanitize surgical or meat preparation instruments in order to eradicate residual prions. Disinfectant solutions may also be used to sanitize work surfaces or rooms to prevent accumulation or transfer of prions. Yet another embodiment of the present invention is the treatment of human and animal food products and nutritional supplements, such as protein solutions or other compounds, with formulations comprising vibriolysin, to eliminate residual prions. For example, a casein hydrolysate or animal food supplements could be further proteolyzed with vibriolysin to eliminate any possible residual prions. A further embodiment of the present invention is the decontamination of tissues used in human transplantation such as collagen tendons or bone grafts. A nationwide survey in Japan reported forty-three cases of CJD with cadaveric dura transplantation by May 1996 (Nakamura, et al., Neurology 53(1):218-20 (1999)). The risk of prion-infected corneal donor appearing in the donor pool was predicted to be 0.045 cases per year, and increased to 2.12 cases per year for potential corneal donation if the data are corrected for age and for possible infected but asymptomatic CJD patients (Hogan, et al., Cornea 18(1):2-11 (1999)). Thus, formulations of vibriolysin and variants thereof would be beneficial in the decontamination of tissues and organs used for transplantation.

The present invention also provides novel methods and formulations for preventing and treating a variety of prion-related diseases in animals and humans, including, but not limited to, scrapie in sheep, BSE in cattle, and kuru disease, GSS and forms of CJD disease in humans. Furthermore, the bovine spongiform encephalopathy or mad cow epidemic in British cattle and, to a lesser degree, in Europe, would make the invention particularly important in Britain and Europe. Through ingestion of prion-contaminated foods and fluids, prions can infect the FDCs or other blood cells of the reticuloendothelial system and thereby gain access to the body and specific body sites, such as the brain. The method of treating prion-associated diseases in subjects in need thereof, comprises administering formulations of pharmaceutical compositions comprising vibriolysin or variants thereof, together with a pharmaceutically effective carrier. As described above, compositions containing proteases produced by Vibrio have been described in WO 98/55604 and U.S. Pat. Nos. 5,130,250, 5,145,681 and 5,505,943. These, and all other U.S. patents cited herein, are specifically hereby incorporated by reference in their entirety. U.S. Pat. No. 5,130,250 discloses the cloning and expression of neutral protease genes from gram-negative microorganisms such as E. coli or Serratia. U.S. Pat. Nos. 5,145,681 and 5,505,943 disclose compositions of a neutral protease, produced by a Vibrio proteolyticus strain, or expressed by recombinant host cells transformed or transfected with an expression vector which provides for the expression of said neutral protease. These patents also teach compositions comprising mutants of the extracellular and recombinant neutral protease. As described below, such pharmaceutical compositions of vibriolysin can be newly formulated to enhance access of vibriolysin to infectious prions. Also provided are new formulations of vibriolysin wherein enzyme activity can be regulated by a change in conditions, such as pH or proteolytic enzyme cleavage.

Another preferred embodiment of the invention provides for a method of treating prion-related diseases during the preclinical stage of the disease, wherein prions incubate within the FDCs before infecting the brain. At the preclinical stage, vibriolysin or variants thereof taken up by the prion-infected cells can cleave prions incubating within FDCs or other cells. Thus, once vibriolysin or variants thereof gain access to the lysosomes of the prion-infected cells, the enzymes degrade the prion proteins and delay or prevent the onset of prion-related diseases, such as CJD. Another preferred embodiment is a method of treating prion-related diseases during the advanced stages of the disease, wherein prions have infected the neurons and brain cells.

Compositions and Formulations

The present invention provides for a composition or formulation comprising one or more vibriolysin or a variant thereof. The compositions or formulations are suitable for any of the methods taught in this disclosure. Preferably, the amount of vibriolysin or a variant thereof comprise 0.0001% to about 10% by weight of the composition of formulation. The concentration of vibriolysin is preferably at least 0.01 μg/ml. More preferably, the concentration of vibriolysin is at least 1 μg/ml. Even more preferably, the concentration of vibriolysin is at least 10 μg/ml. Even much more preferably, the concentration of vibriolysin is at least 100 μg/ml. Generally, the concentration of vibriolysin is from 1-1,000 μg/ml.

This composition or formulation comprises vibriolysin or a variant thereof in a state whereby vibriolysin is not found in nature. The vibriolysin can be isolated or purified from a natural source. The vibriolysin can also be obtained or used from a crude extract of a natural or man-made source. Natural sources of virbiolysin can be bacteria that produce virbiolysin. Man-made sources of vibriolysin are in vivo or in vitro expression systems that produce vibriolysin or a variant thereof. In vivo expression systems can be a microorganism, such as bacteria or yeast, or any organism that does not express vibriolysin in nature.

The use of the composition and formulation can result in at least a 10-fold reduction in the number of infective prion particles. Preferably, the reduction is at least a 100-fold reduction. More preferably, the reduction is at least a 1,000-fold reduction. Even more preferably, the reduction is at least a 10,000-fold reduction. Even much more preferably, the reduction is at least a 1,000,000-fold reduction.

The composition or formulation can be for pharmaceutical or non-pharmaceutical purposes. The compositions or formulations can further comprise another non-vibriolysin protease, either able or unable to cleave prion proteins. Examples of non-vibriolysin protease include, but are not limited to, α-aminoacylpeptide hydrolase, peptidylamino acid hydrolase, acylamino hydrolase, serine carboxypeptidase, metallocarboxypeptidase, thiol proteinase, carboxylproteinase and metalloproteinase. Other examples of protease are serine, metallo, thiol and acid protease, and endo and exo-proteases. Further examples are trypsin, chymotrypsin, and subtilisin. When protease, other than virbiolysin or a variant thereof, is present in the composition or formulation, then conditions of use and the components of the composition or formulation are such that the protease is able to cleave prion or non-prion or a fragment of a prion protein.

The compositions or formulations that are not used for pharmaceutical purposes do not necessarily have to be sterile and can contain cellular debris or components from cells that were used to produce vibriolysin or a variant thereof.

The composition or formulation can further comprise one or more detergents, cleaning agents or cleaning adjunct materials that do not reduce the activity or shelf-life of vibriolysin and also provide further cleaning and/or decontamination function. Suitable adjuncts are of a type and concentration that do not reduce the effectiveness of vibriolysin, or a variant thereof, in reducing the activity of infectious prions. Examples of cleaning adjunct materials include, but are not limited to, detergents, surfactants, solvents, buffers, enzymes, soil release agents, clay soil removal agents, dispersing agents, enzyme stabilizers, builders, bleaching agents, dyes, perfumes, and mixtures. Examples of suitable types of detergent are alkyldimethylamine oxides, alkyl glucosides, alkyl maltosides, alkyl sulfates (such as sodium dodecyl sulfate (SDS)), alkyl thioglucosides, betaines, Big CHAP series, bile acids, CHAP series, digitonin, glucamides, lecithins/lysolecithins, nonionic polyoxyethylene-based detergents (such as TRITON, TWEEN, BRIJ, GENAPOL and THESIT), quaternary ammonium compounds, and the like. Suitable detergents can be determined using routine experimentation (see Neugebauer, J., A Guide to the Properties and Use of Detergents in Biology and Biochemistry, Calbiochem-Novabiochem Corp., La Jolla, Calif., 1988). Detergents can help enhance the effectiveness of vibriolysin by increasing solubilization. The form of the composition or formulation can be a liquid, granule, powder, bar, paste, spray, tablet, gel, form, or the like.

The composition or formulation can further comprise one or more compounds, agents, chemical, molecules, or buffers, alone or in combination, to enhance the self-life of the composition or formulation. Under alkaline conditions, the biological activity of vibriolysin or a variant thereof can be maintained for long periods. Preferably, the time period is at least 6 months. More preferably, the time period is at least one year. Even more preferably, the time period is at least two years. Even further more preferably, the time period is at least three years. Even further more preferably, the time period is at least five years. Further, the efficacy, or the enzymatic or biological activity, of the vibriolysin or a variant thereof is able to persist for long time periods after application, contact or administration. This means that a sufficient ability to reduce or destroy the infectivity of prions persists. The efficacy is able to persist for at least one day after application, contact or administration. Preferably, it is able to persist for at least two days. More preferably, it is able to persist for at least three days. Even more preferably, it is able to persist for at least one week. This persistence of vibriolysin or a variant thereof in shelf-life and application is due to its property of not autolysing. By way of contrast, Proteinase K is expensive and unstable and is not as suitable, even if it were 10-fold more potent. The vibriolysin or variants thereof of the present invention can be used over and over by the user.

Regarding the alkalinity of the composition or formulation, the pH of the composition or formulation can be from 7, or more than 7 to 8, 8-10, 10-12, or 12-14. Preferably it has a pH greater than 7. More preferably, the pH is from pH 8 to 14. Even more preferably, the pH is from pH 8 to 12.

The composition or formulation can be used for any object that requires the reduction of the infectivity or destruction of prions present. These objects include actual or potentially prion-contaminated facilities and instruments. These facilities and instruments especially include facilities and instruments that (1) have a likelihood of contamination by prions, and/or (2) have a need not to be contaminated by prions in order to avoid or reduce infection of humans or livestock. The object can be a room, facility, instrument, foodstuff, animal feed, or the like. The object can be any hardsurface or comprise one or more hardsurfaces. Preferably, these objects are those that have a high likelihood to come in contact with animals, such as livestock, such as cattle, and humans. More preferably, these objects are those may be ingested, absorbed or come into contact with the mucous surfaces of animals and/or humans. Examples of rooms and facilities are any that may be entered by an animal or human, especially any used for medical or surgical purposes, or any involved with the slaughtering of animals or livestock, or used for handling foodstuff or animal feed. Examples of instruments are dental instruments, medical and surgical instruments, utensils, cutlery, abattoir/slaughterhouse/butcher equipment, and the like. The composition or formulations are also used for decontaminating and/or disinfecting and/or cleaning instruments and facilities involved in research where prions are utilized, or have a high likelihood of coming in contact with prion proteins.

For treatment at the preclinical stage, pharmaceutical compositions of vibriolysin are formulated to enhance the enzyme's capability to follow the prion infectious path. As described above, infectious prion particles may enter the body by ingestion of prion-contaminated food products. Prions ingested orally are taken up into the FDCs of the germinal centers within the reticuloendothelial system. Prions may bind to complement factors and the resulting prion-complement factor complex would then be captured by FDCs though complement receptors such as CR1/2. These cells normally function to capture native antigen and form immune complexes for presentation to B cells. Thus, the complement system may play an important role in opsonizing prion particles and enhancing complement receptor-mediated uptake into the FDCs. Prions in the FDCs may then incubate preclinically, increasing in titer and concentration before being transported to the brain by FDCs or some type of white blood cell. Thus, when prions enter the body via a peripheral route, they replicate in the lymphoreticular tissues before moving through the nerves into the spinal cord or brain stem, and then to the brain.

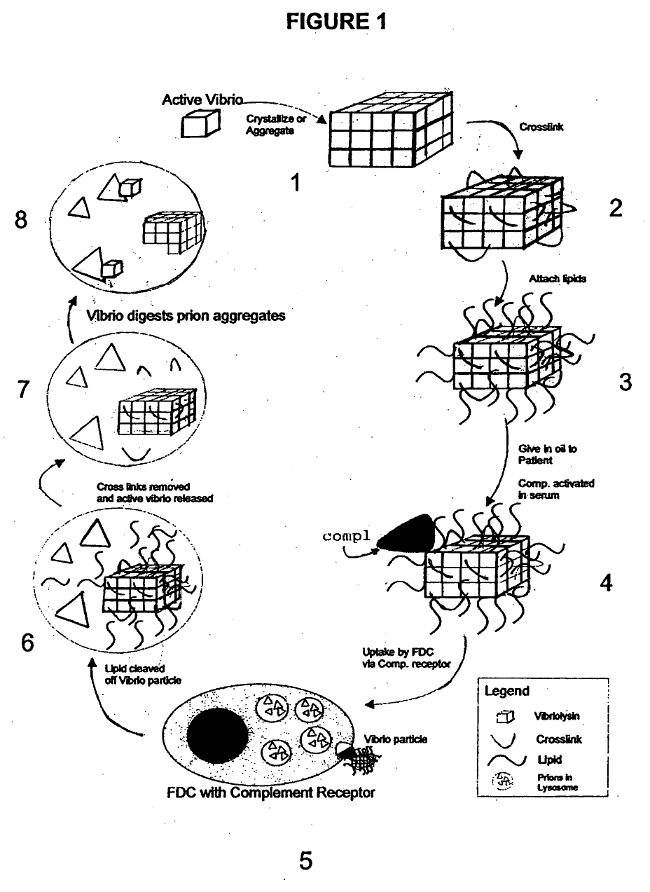

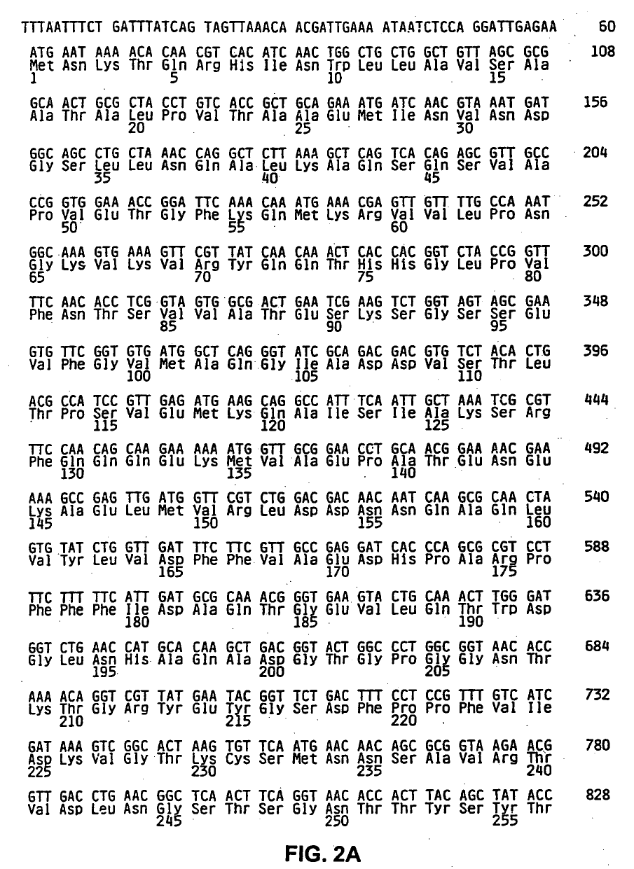

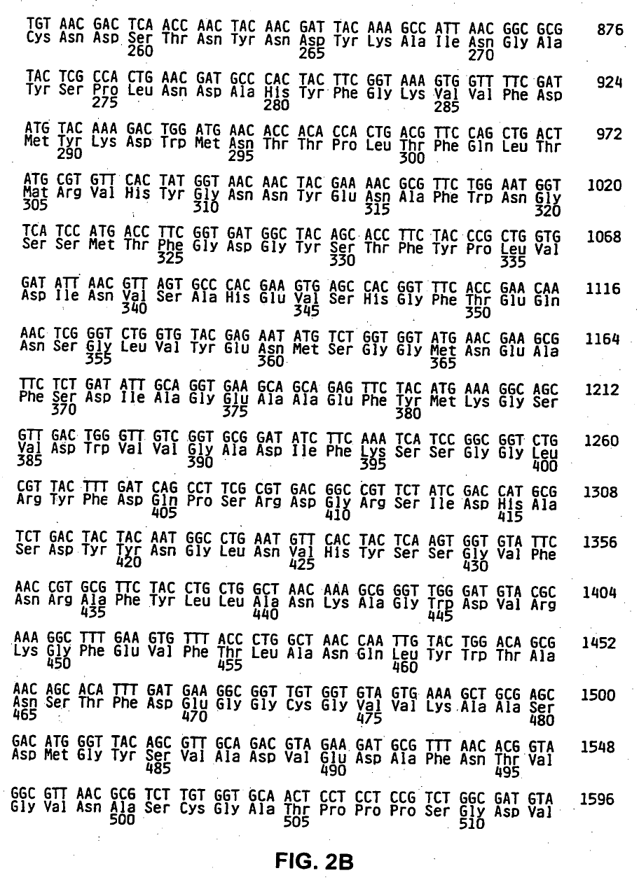

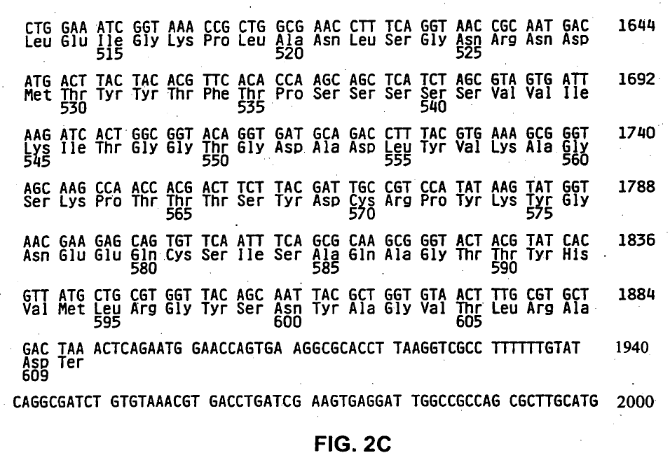

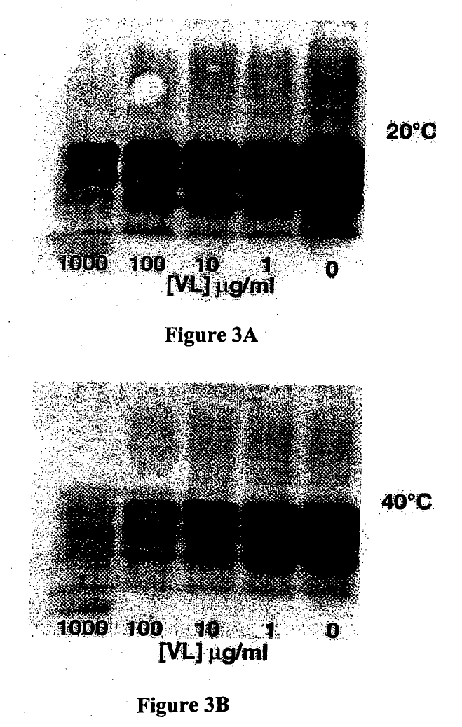

The present invention provides a formulation of a pharmaceutical composition comprising vibriolysin or a variant thereof, wherein the enzyme is formulated into a structure resembling the infectious prion particle (FIG. 1). Such a structure can have a crystallized, particulate, lipid conjugated, or liposomal form, which enhances the enzyme's capability to track the prions' infectious path and to access target sites which accumulate prions within the body. For example, vibriolysin or a variant thereof is processed into a microcrystalline or equivalent form and stabilized with crosslinking to temporarily maintain insolubility of the enzyme and enzyme inactivation at normal pH (7.0-7.4). The microcrystalline structure is then lipidated, wherein fatty acids are attached to the surface of the crystal, to create an “organism”-like structure. The lipid coat would enhance uptake with fats by the lacteals in the intestines and entry of the particles into the circulation by way of the thoracic duct, the lymph system that normally moves fat from the bowel to the bloodstream. U.S. Pat. Nos. 5,618,710; 5,849,296; 5,976,529; and 6,011,001 disclose protein crystals crosslinked with a multifunctional crosslinking agent and having resistance to exogenous proteolysis. Such proteins may be enzymes or antibodies. U.S. Pat. No. 5,849,296 also discloses a lipase crystal crosslinked with a multifunctional crosslinking agent and having resistance to exogenous proteolysis. U.S. Pat. Nos. 5,976,529 and 6,011,001 describe methods for carrying out protein and enzyme therapy by administering orally a crosslinked enzyme crystal. U.S. Pat. No. 6,042,824 describes methods for producing crosslinked protein crystal formulations and methods for using them to optimize chemical reactions in organic solvents, including those used in industrial scale chemical processes. Such methods are useful in formulating vibriolysin and variants thereof into prion-like structures.

A further embodiment is a formulation wherein the prion-like structure is modified by coating particle with a lipid surface to enhance complement activation as is observed in liposomes. As described above, the opsonization of the particles by the complement proteins results in their uptake into the FDC's of the germinal centers. After uptake into the lysosomes of the FDCs, the lipid coat is degraded by the acid lipases and the vibriolysin enzyme dissolved within the lysosomal milieu.

For treatment at the advanced stage, pharmaceutical compositions of vibriolysin are formulated to enhance the enzyme's capability of uptake into the neurons and across the blood brain barrier. As described above, the vibriolysin proteins and variants thereof are formulated into a structure resembling a prion, enabling the enzyme to track the path of the prion from the lymphoreticular tissues moving through the nerves into the spinal cord or brain stem, and then to the brain. Entry into the brain across the blood brain barrier is normally restricted to small hydrophobic molecules, specifically transported nutrients such as glucose and certain amino acids, and specifically transcytosed macromolecules such as transferrin (Staddon and Rubin, Curr. Opin. Neurobiol. 6: 622-627 (1996)). The present invention provides a formulation, in which the vibriolysin structure is further engineered to access passive carrier and active transport systems across the blood-brain barrier and across neuronal cell membranes. The blood brain barrier permeability to vibriolysin may be enhanced by: (1) modification of the vibriolysin structure to increase its lipid solubility; (2) linkage of the vibriolysin molecule to a peptide from a transporter system using liposome linkers or nanoparticle technology (C & EN, Sep. 18, 2000, page 58); and (3) administration with agents shown to increase blood brain barrier permeability, including bradykinin B₂ receptor agonists, serotonin, and H₂ receptor agonists (U.S. Pat. Nos. 5,112,596 and 5,268,164; Emerich, et al., Br. J. Cancer 80:964-70; Mackie, et al., Pharm. Res. 16:1360-1365 (1999); Wahl, et al., Immunopharmacology 33:257-263 (1996); Morel, et al., Inflammation 14(5):571-583 (1990); Abbott, Cell. Mol. Neurobiology 20:131-147 (2000); Mashito, et al., Immunopharmacology 43:249-253 (1999)).

Routes of Administration

For use as a therapeutic, the activity of the vibriolysin protease is restricted to the lysosome in order to prevent the degradation of blood clotting components while in the circulation. Hence, another embodiment of the present invention is a formulation of vibriolysin capable of uptake into lysosomes. The uptake of vibriolysin into lysosomes may be enhanced by: (1) complement-activating surface characteristics; (2) tagging the vibriolysin protein with a lysosomal enzyme, such as alpha-L-iduronidase, which is normally taken up by mannose-6-phosphate receptors and targeted to lysosomes; and (3) attachment of mannose-6-phosphate residues of sufficient quantity or addition of mannose sugars or proteins with mannose in linkage to target macrophages of the reticuloendothelial system, in order to increase the enzyme binding affinity and uptake by mannose-6-phosphate receptors.

A further embodiment of the present invention is a formulation of vibriolysin wherein its enzyme activity can be regulated by changes in conditions such as pH or by lysosomal enzymes. U.S. Pat. No. 6,140,475 discloses a method for controlled dissolution of crosslinked protein crystals. The method comprises producing crosslinked protein crystals with a multifunctional crosslinking agent, wherein the resulting protein crystals are characterized by the ability to change from an insoluble and stable form to a soluble and active form upon a change in the environment, including changes in temperature, pH, chemical composition, concentration, and shear force. The multifunctional crosslinking agent is glutaraldehyde at a concentration of between 0.076% and about 0.05% (vol/vol). International Patent Application No. WO 9955310 discloses formulations and compositions of protein or nucleic acid crystals, methods of crystallization of proteins and nucleic acids, methods of stabilization of protein and nucleic acid crystals, and methods of encapsulating proteins, glycoproteins, enzymes, antibodies, hormones, and peptide crystals or crystal formulations into compositions for biological delivery to humans and animals. Thus, the vibriolysin enzyme is administered in a microcrystalline, lipidated prion-like form that is inactive, but can become activated only upon exposure to specific coriditions, such as a change in pH, temperature or lysosomal enzyme composition or concentration within the lysosome. The activity or half-life of vibriolysin can also be restricted by engineering the enzyme to impart a sensitivity to degradation by other lysosomal proteases or denaturation under specific conditions, such as an acidic environment, within a reasonable time period, and thereby prevent excess vibriolysin activity and any potential destruction of the lysosome and cell death.

Therapeutic enzymes may be administered in a number of ways such as parenteral, topical, intranasal, inhalation or oral administration. In some embodiments, the invention provides for administering the enzyme in a pharmaceutical composition together with a pharmaceutically-acceptable carrier, which may be solid, semi-solid or liquid or an ingestible capsule. Examples of pharmaceutical compositions useful in the present invention include tablets and drops, such as nasal drops. Compositions for topical application include, but are not limited to, ointments, jellies, creams and suspensions, aerosols for inhalation, nasal spray, and liposomes.

To produce pharmaceutical compositions for oral application containing the therapeutic enzyme(s), the enzyme(s) may be mixed with a solid, pulverulent carrier. The carrier may include, but is not limited to, lactose, saccharose, sorbitol, mannitol, starch (for example, a potato starch or a corn starch), amylopectin, laminaria powder, citrus pulp powder, cellulose derivative, and gelatine. The pharmaceutical compositions may also include lubricants such as magnesium or calcium stearate or a Carbowax or other polyethylene glycol waxes, and they may be compressed to form tablets or cores for dragees. If drakes are required, the cores may be coated with, for example, a concentrated sugar solution. The sugar solutions may contain gum arabic, talc and/or titanium dioxide, or alternatively, a film-forming agent dissolved in easily volatile organic solvents or mixtures of organic solvents. Dyestuffs may be added to such coatings, for example, to distinguish between different contents of active substance. For a composition of soft gelatine capsules consisting of gelatine, or glycerol as a plasticizer, or similar closed capsules, the active substance may be admixed with a Carbowax® or a suitable oil such as sesame oil, olive oil or arachis oil. Hard gelatine capsules may contain granulates of the active substance with solid, pulverulent carriers such as lactose, saccharose, sorbitol, mannitol, starches (for example, potato starch, corn starch or amylopectin), and cellulose derivatives or gelatine, and they may also include magnesium stearate or stearic acid as lubricants.

Therapeutic enzymes of the present invention may also be administered parenterally such as by subcutaneous, intramuscular or intravenous injection or by sustained release subcutaneous implant. In subcutaneous, intramuscular and intravenous injection, a therapeutic enzyme or other active ingredient may be dissolved or dispersed in a liquid carrier vehicle. For parenteral administration, the active material may be suitably admixed with an acceptable vehicle, preferably of the vegetable oil variety, such as peanut oil, cottonseed oil and the like. Other parenteral vehicles such as organic compositions using solketal, glycerol, formal, and aqueous partnered formulations may also be used. For parenteral application by injection, compositions may comprise an aqueous solution of a water-soluble pharmaceutically-acceptable salt of the active acids according to the invention, desirably in a concentration of 0.5-10%, and optionally also a stabilizing agent and/or buffer substances in aqueous solution. Dosage units of the solution may advantageously be enclosed in ampoules. When therapeutic enzymes are administered in the form of a subcutaneous implant, the compound may be suspended or dissolved in a slowly dispersed material known to those skilled in the art or administered in a device which slowly releases the active material through the use of a constant driving force, such as an osmotic pump. In such cases, administration over an extended period of time may be possible.

For topical application, the pharmaceutical compositions are suitable in the form of an ointment, gel, suspension, cream, or the like. The amount of active substance may vary, for example, between 0.05-20% by weight of the active substance. Such pharmaceutical compositions for topical application may be prepared in known manners by mixing the active substance with known carrier materials, including but not limited to, isopropanol, glycerol, paraffin, stearyl alcohol, or polyethylene glycol. The pharmaceutically-acceptable carrier may also include a known chemical absorption promoter. Examples of absorption promoters are dimethylacetamide (U.S. Pat. No. 3,472,931), trichloro ethanol or trifluoroethanol (U.S. Pat. No. 3,891,757), and certain alcohols and mixtures thereof (British Patent No. 1,001,949). A carrier material for topical application to unbroken skin is also described in the British Patent Specification No. 1,464,975, which discloses a carrier material consisting of a solvent comprising 40-70% (v/v) isopropanol and 0-60% (v/v) glycerol, the balance, if any, being an inert constituent of a diluent not exceeding 40% of the total volume of solvent.

The dosage at which pharmaceutical compositions containing enzymes are administered may vary within a wide range and depend on various factors, such as the severity of the infection and the age of the patient. The dosage may have to be individually adjusted. The pharmaceutical compositions containing a therapeutic enzyme may suitably be formulated so that they provide doses within these ranges either as single dosage units or as multiple dosage units. In addition to containing a therapeutic enzyme, the pharmaceutical compositions may contain one or more substrates or cofactors for the reaction catalyzed by the therapeutic enzyme in the compositions.

The present invention provides a DNA sequence encoding vibriolysin or a variant that can be used in a transgenic animal or as gene therapy to treat prion-associated diseases. The therapeutic enzymes, according to the present invention, may be administered by means of transforming patient cells with nucleic acids encoding a therapeutic enzyme when the therapeutic enzyme is a protein or ribonucleic acid sequence. A nucleic acid sequence encoding a therapeutic enzyme may be incorporated into a vector for transformation into cells of a subject to be treated. A vector may be designed to integrate into the chromosomes of the subject, for example, retroviral vectors, or to replicate autonomously in the host cells. Vectors containing nucleotide sequences encoding a therapeutic enzyme may be designed to provide for continuous or regulated expression of the enzyme. Additionally, the genetic vector encoding the therapeutic enzymes may be designed to stably integrate into the cell genome or to only be present transiently. The general methodology of conventional genetic therapy may be applied to polynucleotide sequences encoding therapeutic enzymes. Reviews of conventional genetic therapy techniques can be found in Friedman, Science 244:1275-1281 (1989); Ledley, J Inherit. Metab. Dis. 13:587-616 (1990); and Tososhev, et al., Curr Opinions Biotech. 1:55-61 (1990).

A preferred embodiment is a recombinant vibriolysin protein or variant thereof, which can specifically target and gain access to cells such as FDCs known to accumulate prions. Another preferred embodiment is a recombinant vibriolysin protein or variant thereof, wherein its enzymatic activity, such as the transformation between active and inactive forms, is regulated by specific conditions, such as exposure to infectious prion particles, a specific pH range, or proteolytic enzymes. Yet another preferred embodiment is a recombinant vibriolysin protein or variant thereof that exists in an inactive “pro-form”, which becomes activated by enzymatic cleavage, such as by lysosomal enzymes.

### EXAMPLE

Prions or infection proteins derived animal forms of prion disease (scrapie in sheep, bovine spongiform encephalopathy BSE) in cows) as well as the human form of the disease (variant Creutzfeld-Jacob Disease (vCJD)) have the special property of being resistant to protease digestion. This property is believed in part to be an important characteristic in their propagation and the difficulty in eliminating prions from the body. Prions are composed of very hydrophobic prion proteins that form large aggregates. Vibriolysin, a protease with the propensity to cleave hydrophobic regions of proteins, is a likely candidate for a protease that can cleave the prion protein. The following experiment was undertaken to assess the ability of vibriolysin to cleave prion proteins.

Methods and Materials

A sample of vCJD affected brain was obtained and a 20% homogenate prepared in phosphate buffered saline. The homogenate was dilute to a 10% w/v solution with Tris-HCL and NaCl solutions to give a final homogenate of 10% vCJD brain, 100 mM Tris-HCl, and 150 mM NaCl, pH 8.0. Increasing concentrations of vibriolysin were added to aliquots of the 10% brain homogenate and the mix incubate at either 20° C. (FIG. 3A) or 40° C. (FIG. 3B). After 1 hour, the reactions were stopped by addition of SDS-PAGE loading buffer containing 8 mM AEBSF. The samples were boiled at 100° C. for 10 mins and the tubes spun to collect the condensate. The samples were run on standard SDS-PAGE gels, the proteins transferred to PVDF using a standard Western blot method. The Prion protein was visualized using a mouse monoclonal antibody IgG2 (ICSM35). The primary antibody was developed using a goat anti-mouse secondary antibody conjugated to horse radish peroxidase and chemiluminescent detection performed.

Results

Digestion of the prion in human vCJD brain shows that Vibriolysin can cleave the prion protein over the one hour period, particularly at the highest concentration of 1 mg/ml. Given that the conditions tested are not the optimal ones for Vibriolysin, it would be expected that even higher potency would be possible at higher pH's and temperature. The data show that Vibriolysin has a prion-digesting activity that may be useful in the disinfection of surgical instruments or for other purposes. In addition, the unique stability, pH properties and lack of autodigestion, make Vibriolysin an optimal enzyme for the commercial application of proteases to disinfection and decontamination of prions.

The invention, and the manner and process of making and using it, are now described in such full, clear, concise, and exact terms as to enable any person skilled in the art to which it pertains, to make and use the same. It is to be understood that the foregoing describes preferred embodiments of the present invention and that modifications may be made therein without departing from the spirit or scope of the present invention as set forth in the claims. To particularly point out and distinctly claim the subject matter regarded as invention, the following claims conclude this specification.

1. A composition capable of reducing the infectivity of prion, comprising vibriolysin or a variant thereof in an amount effective to reduce the infectivity of prion, and one or more cleaning adjunct materials.

2-3. (canceled)

4. The composition according to claim 1, wherein, after one year of the manufacture of said composition, said composition is capable of retaining its capacity of reducing the infectivity of prion.

5-6. (canceled)

7. A method of reducing the activity of infectious prions, comprising: contacting a vibriolysin protease or variant thereof with said prions in an amount effective to cleave or degrade said prions or destroy their infective activity.

8. (canceled)

9. A method of sanitizing facilities or instruments contaminated with pirons comprising: contacting said prions with a solution comprising a vibriolysin protease or variant thereof in an amount effective to reduce or eradicate prion contamination of said instruments and facilities.

10. The method of claim 9, wherein the solution contains a detergent.

11. A pharmaceutical composition comprising a DNA sequence encoding vibriolysin or a variant thereof, wherein said vibriolysin or a variant thereof has the ability to gain access to target sites that accumulate prions within the body, and a pharmaceutical carrier.

12. A pharmaceutical composition comprising a DNA sequence encoding vibriolysin or a variant thereof, wherein said vibriolysin or a variant thereof has the ability to transform from an inactive state to an activated state capable of cleaving prions and a pharmaceutical carrier.

13. A formulation of the pharmaceutical composition comprising vibriolysin or a variant thereof, wherein said formulation is a prion-like structure, capable of tracking the infectious path of the prion from the lymphoreticular tissues to the nerves, spinal cord, brain stem, or brain.

14. The formulation according to claim 13, wherein said prion-like structure comprises vibriolysin crystals crosslinked with a multifunctional crosslinking agent and having resistance to exogenous proteolysis and a coat of lipid chains.

15. The formulation according to claim 13, wherein the prion-like structure is modified to increase access of said structure to one or more target sites that accumulate prions within the body.

16. The formulation according to claim 15, wherein said one or more target sites comprises follicular dendritic cells.

17-20. (canceled)

21. The formulation according to claim 13, wherein said prion-like structure is modified by attachment of moiety to enhance uptake by mannose receptors and transfer to the lysosomes.

22. The formulation according to claim 13, wherein said prion-like structure is modified to increase passage of said structure across the blood brain barrier into the central nervous system.

23. The formulation according to claim 13, wherein said prion-like structure comprises vibriolysin or a variant thereof capable of transforming from an inactive state to an activated state, wherein said vibriolysin or variant thereof is capable of cleaving prions upon a change in environment.

24. The formulation according to claim 23, wherein said change in environment is a change in pH, temperature, concentration, or chemical composition.

25. A method of treating or preventing a prion-caused, prion-related or prion-like diseases in a subject or biological tissue in need thereof comprising: administering to said subject a formulation of a pharmaceutical composition comprising a vibriolysin protease or variant thereof with a pharmaceutically acceptable carrier in an amount effective to treat or prevent said prion-caused, prion-related or prion-like diseases.

26. The method according to claim 25, wherein said prion-related disease is selected from the group consisting of scrapie, bovine spongiform encephalopathy, kuru disease, Gerstmann-Straussler-Scheinker Syndrome, and Creutzfeld-Jacob disease.

27-29. (canceled)

30. The method according to claim 25, wherein the prion-like structure is modified to increase access of said structure to one or more target sites that accumulate prions within the body.

31. The method according to claim 30, wherein said one or more target sites comprises follicular dendritic cells.

32. The method according to claim 30, wherein said one or more target sites comprises lysosomes.

33-39. (canceled)
